(19) United States
(12) Patent Application Publication
(10) Pub. No.: US 2007/0038101 A1
(43) Pub. Date: Feb. 15, 2007

(54) Method of forming strain images

(75) Inventors: Ra Young Yoon, Seoul (KR); Sung Jae Kwon, Seoul (KR); Moo Ho Bae, Seoul (KR); Mok Kun Jeong, Seoul (KR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Medison Co., Ltd., Hongchun-gun (KR)

(21) Appl. No.: 11/434,890

(22) Filed: May 17, 2006

(30) Foreign Application Priority Data
Jul. 27, 2005 (KR) 10-2005-68251
Nov. 16, 2005 (KR) 10-2005-109477

(51) Int. Cl. A61B 8/00

(52) U.S. Cl. 600/443

(57) ABSTRACT

There is provided a method of forming strain images by decreasing the decorrelation of receive signals, which vary with time or space. More specifically, the decorrelation between the receive signals obtained with and without applying stress to a target subject is reduced to decrease an error, which occurs during the calculation of a delay. Also, a center frequency, which varies according to a depth of the target subject, is compensated to form the strain image.

### FIELD OF THE INVENTION

The present invention generally relates to a method of forming strain images in an ultrasound imaging system, and more particularly to a method of forming strain images by decreasing the decorrelation of ultrasound receive signals.

### BACKGROUND OF THE INVENTION

An ultrasound imaging system is widely used in the medical field. In the ultrasound imaging system, ultrasound signals are transmitted to the tissues of a target subject (e.g., humans), wherein the ultrasound signals reflected from the tissues are transformed into receive signals. An ultrasound image is formed by using the receive signals.

The ultrasound image is mainly expressed with a brightness-mode (B-mode) based on reflection coefficients, which vary according to the differences in impedance between the tissues of the target subject. However, it is difficult to observe a lesion, such as a tumor or carcinoma, with the B-mode image. This is because the reflection coefficients of the tumor or carcinoma are not so different from those of adjacent tissues.

The eleastrography for forming ultrasound elasticity images (i.e., strain images) utilizes the mechanical characteristics of the tissues, which are difficult to be observed in the B-mode image. Thus, the eleastrography can be of great use in diagnosing lesions.

The mechanical characteristics of the tissues can be obtained by comparing a first receive signal and a second receive signal, which are obtained without and with the application of stress (i.e., force per unit area), respectively, to the target subject. The first and second receive signals have a RF form.

Due to the stress, each tissue has a different displacement, which reflects the mechanical characteristics (e.g., tissue hardness). The displacement can be obtained by considering the phase difference or the delay between the first and second receive signals. In the eleastrography, the displacement of each tissue can be obtained by computing a cross correlation or an autocorrelation of the first and second receive signals. In the cross correlation, the first and second receive signals in the RF form are computed. However, in the autocorrelation, the first and second receive signals should be converted into I/Q baseband signals.

The autocorrelation has the advantage of enhancing the speed of computation since the data amount of the I/Q baseband signals is less than that of the RF signals. However, the autocorrelation expresses the displacement in terms of phase value. Thus, an additional step is required to convert the phase value into a time value.

A center frequency of ultrasound transmission signals is used to convert the phase value into the time value. The center frequency varies according to the depth of tissues in the target subject. Consequently, an error will occur if a fixed value is used as the center frequency during the conversion of the values.

In case the phase value is computed by using the autocorrelation, aliasing is generated when the phase difference of first and second receive signal is greater than ½ wavelength of the ultrasound transmission signals. Therefore, an additional process should be introduced to compensate for the aliasing.

As the depth of the tissues increases, the first and second receive signals become increasingly different in terms of phase and shape. Thus, there is a greater chance for error due to the decorrelation of the first and second receive signals.

### SUMMARY OF THE INVENTION

It is, therefore, the present invention provides a method of forming strain images by decreasing the decorrelation of the receive signals obtained without and with the application of stress to a target subject. More specifically, the present invention provides a method of forming real-time medical images by decreasing the decorrelation of the receive signals. Additionally, the present invention provides a method of forming ultrasound elasticity images by compensating the variation in center frequency of ultrasound transmission signals.

According to the present invention, there is provided a method of forming a strain image, comprising: obtaining a first receive signal and a second receive signal, wherein the second receive signal is delayed with respect to the first receive signal; computing a first correlation of the first receive signal and the second receive signal for a sampling period; computing an instantaneous frequency based on the first correlation; estimating a delay amount between the first and second receive signals; selecting one of the first and second receive signals and shifting the selected signal as much as the estimated delay amount to reduce a delay between the first and second receive signals, wherein the non-selected signal remains as a non-shifted signal; computing a second correlation of the non-shifted signal and the shifted signal; obtaining a delay between the first and second receive signals based on the instantaneous frequency, the estimated delay amount and the second correlation; obtaining a strain based on the delay; and forming a strain image based on the strain.

According to the present invention, there is provided a method of forming strain images, comprising the steps of: transmitting an ultrasound signal to a target object without or with an application of stress to the target subject; obtaining a first receive signal and a second receive signal, wherein the first receive signal is obtained without applying the stress to the target subject and the second receive signal is obtained with applying the stress to the target subject; computing a first correlation of the first receive signal or the second receive signal for a sampling period; computing an instantaneous frequency based on the first correlation; estimating a delay amount between the first and second receive signals; selecting one of the first and second receive signals and shifting the selected signal as much as the estimated delay amount to a direction of reducing a delay between the first and second receive signals, wherein the non-selected signal remains as a non-shifted signal; obtaining a second correlation of the shifted signal and the non-shifted signal; obtaining a delay between the first and second receive signals based on the instantaneous frequency, the estimated delay amount and the second correlation; obtaining a strain based on the delay; and forming a strain image based on the strain.

### BRIEF DESCRIPTION OF THE DRAWINGS

The above and other objects and features in accordance with the present invention will become apparent from the following descriptions of preferred embodiments given in conjunction with the accompanying drawings, in which:

FIG. 1 illustrates a graph of first and second receive signals obtained without and with the application of stress, respectively, to a target subject;

FIG. 2 is a schematic diagram of a delay signal model for computing a delay;

FIG. 3a shows the first and second receive signals in adjacent two windows;

FIG. 3b shows the first receive signal and the second receive signal shifted as much as an estimated delay amount; and

FIG. 4 is a flow chart showing a method of forming an ultrasound elasticity image according to the present invention.

### DETAILED DESCRIPTION OF THE PRESENT INVENTION

Hereinafter, the embodiments of the present invention will be described by referring to the attached drawings.

A first receive signal in an RF form is obtained by transmitting ultrasound signals from a probe to a target subject without applying any stress to the tissues of the target subject. A second receive signal, which is also in the RF form, is obtained with applying the stress to the tissues of the target subject. Along the direction of the stress, the tissues of the target subject move so that the second receive signal is delayed compared to the first receive signal.

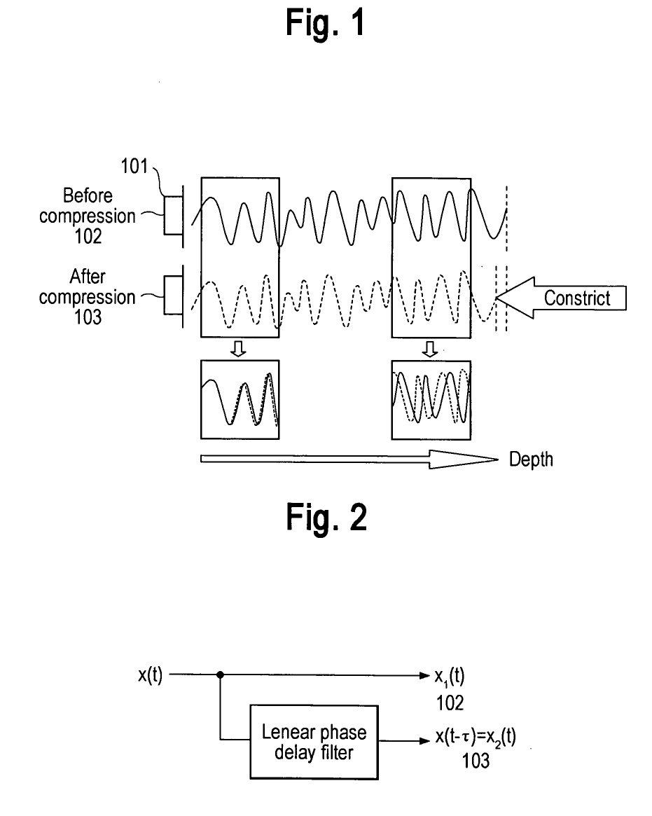

If the center frequency and amplitude of the transmitting ultrasound signal are denoted as ω₀ and r(t), respectively, then the first receive signal and the second receive signal having the RF form can be defined as x₁(t) and x₂(t), as shown in equations 1 and 2 below.
x₁(t)=r(t)cos(ω₀t+φ(t)) (Equation 1)
x₂(t)=r(t−τ)cos(ω₀(t−τ)+φ(t−τ)) (Equation 2)
In equations 1 and 2, ‘φ(t)’ denotes the phase which varies with time, and ‘τ’ denotes the delay due to the stress. FIG. 1 shows the shapes of the first and second receive signals. As shown in FIG. 2, the delay τ due to the stress is modeled with an all pass filter having a linear delay.

The delay of the second receive signal is smaller at the region near the probe compared to other regions that are far from the probe. This is because the phase difference of the first and second receive signals near the probe is relatively small.

The displacement of each tissue can be calculated with the delay of the second receive signal. The degree of the displacement of the tissue depends on the hardness of the tissues when a constant stress is applied to the tissues in one direction. Accordingly, a distortion ratio, i.e., strain can be obtained by calculating the displacement varying with the stress and by obtaining the derivative of displacement between the first and second receive signals. The elasticity ultrasound image (i.e., strain image) is formed on the basis of the strain.

In order to calculate the correlation of the first and second receive signals with autocorrelation, the first and second receive signals in the RF form are demodulated and converted into I/Q baseband signals. Further, the delay is computed to obtain the phase difference of the first and second receive signals. By demodulating, the first and second receive signals can be converted into I/Q baseband signals, as shown by equations 3 and 4 below.
x₁(t)=r(t)e^{jφ(t)} (Equation 3)
x₂(t)=r(t−τ)e^{j(-ω}^{₀}^{τ+φ(t-τ))} (Equation 4)
The phase difference ΔΦ between the first and second receive signals can be obtained by computing the correlation of the first receive signal x₁(t) and the second receive signal x₂(t), as expressed in equation 5 below.
ΔΦ=arg<x₁·x₂*>=ω₀τ+φ(t)−φ(t−τ) (Equation 5)
In equation 5, “<·>” denotes the function for computing the correlation operation and “arg<·>” denotes the function for obtaining the phase. The first term of the Taylor series expansion of φ(t−τ) is expressed as equation 6, which is shown below.
φ(t−τ)≅φ(t)−τφ′(t) (Equation 6)

By applying equation 6 to equation 5, the phase difference ΔΦ can be approximated as equation 7, which is shown below.
ΔΦ=ω₀τ+τφ′(t) (Equation 7)
The following equation 8 is obtained by rearranging the equation 7 with respect to the delay τ.

$$τ = \frac{ΔΦ}{{ω}_{0} + {ϕ}^{′} ( t )} = \frac{ΔΦ}{{ω}_{0} + {ω}_{B} ( t )} \qquad ( Equation 8 )$$

In equation 8, ‘φ′(t)’ denotes the derivative of the phase, which can be considered as the instantaneous frequency ω_{B} of the first or second receive signal converted into the form of I/Q baseband. For example, the instantaneous frequency ω_{B}, i.e., φ′(t) can be obtained by calculating the correlation of the first receive signals converted into I/Q baseband signals at time t and at time t+T, as shown in equation 9 below.

$${ϕ}^{′} ( t ) = {ω}_{B} ( t ) = \frac{arg 〈 {x}_{1} ( t ) \cdot {x}_{1}^{*} ( t + T ) 〉}{T} \qquad ( Equation 9 )$$

In equation 9, ‘T’ denotes a sampling period.

Referring to equation 8, if ω₀>ω_{B}(t), then the delay τ can be computed approximately as shown in equation 10 below.

$$τ = \frac{Δ Φ}{{ω}_{0}} \qquad ( Equation 10 )$$

An error in the delay will occur if the denominator of the equation 10 is fixed as the center frequency ω₀ of the transmitting signal. This is because the first and second receive signals have wide bandwidths and the frequencies thereof vary according to the depth of the tissues in the target subject. The error can be reduced by using the components of the instantaneous frequency ω_{B} of the first or second receive signal converted into I/Q baseband signal.

When the stress is applied to the target subject, the shape of the second receive signal changes with respect to the first receive signal. The displacement of the target subject increases as the depth of the tissue in the target subject becomes deeper. This is because the phase difference of the first and second receive signals increases in proportional to the depth of the tissue. Therefore, the decorrelation of the first and second receive signals and the error in the delay are relatively large in the deep region.

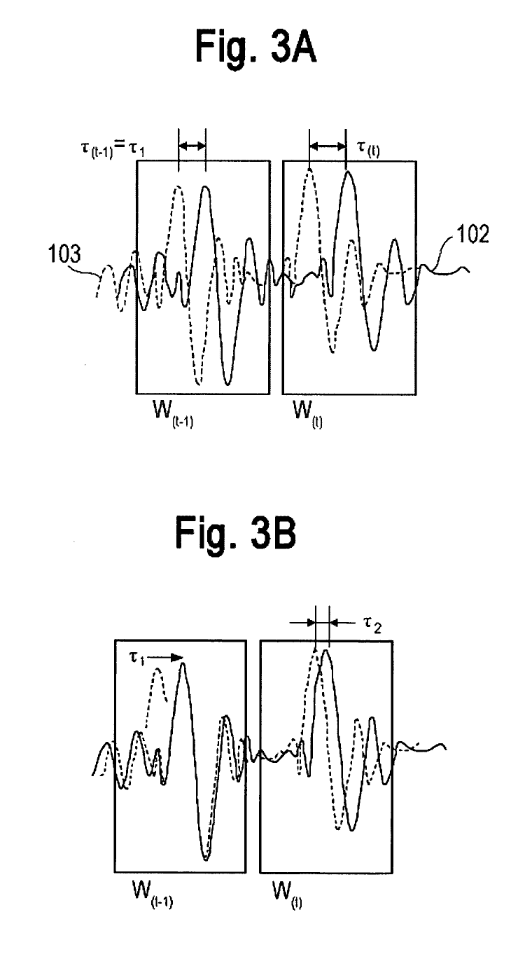

The decorrelation and the error of the deep region can be reduced by determining the delay of the second receive signal in the deep region by using the delay of the second receive region in a shallow region. To this end, a delay amount of the second receive signal in the shallow region is estimated, wherein one of the first and second receive signals is shifted as much as the estimated delay amount such that the delay decreases. Then, the delay between the two receive signals in the deep region is computed. Consequently, the delay of the second signal in the deep region can be obtained by a sum of the estimated delay amount and the computed delay, in which the shift is reflected. The details of these processes will be explained with reference to FIGS. 3a and 3b.

In one embodiment of the present invention, the first and second receive signals are divided into a plurality of windows according to the depth of the tissues in the target subject or the lapse of time. FIGS. 3a and 3b show windows w(t−1) and w(t) dividing the first receive signal 102 and the second receive signal 103. The window w(t−1) and window w(t) correspond to the shallow region and the deep region, respectively.

As can be seen from the comparison of the first and second receive signals in the two windows w(t−1) and window w(t), the phase difference and the delay increase when the depth becomes deeper (i.e., when the lapse of time becomes longer). If the two windows are adjacent to each other, the phase difference and the delay between the first and second receive signals in the two windows are not so significant. Therefore, the aliasing can be effectively prevented, which otherwise occurs when the phase difference is greater than π.

If the delay between the first and second receive signals in the window w(t−1) is τ(t−1)=τ₁, then the estimated delay amount of the second signals is determined as τ₁ in window w(t) adjacent to the window w(t−1).

As shown in FIG. 3b, when the second receive signal is shifted as much as the estimated delay amount τ₁ in accordance with the linear interpolation method, the phase difference between the first receive signal and the shifted second receive signal in the window w(t−1) becomes zero. This is because the two signals are completely overlapped by the shift.

After the shift, the delay τ₂ of the second receive signals in the window w(t) is computed. The computed delay τ₂ in the window w(t) is smaller than the estimated delay amount τ₁. Consequently, the correlation between the first and second receive signals in the window w(t) increases. Therefore, the noise and aliasing decrease due to the reduced delay.

Since the computed delay τ₂ of the second receive signal in the window w(t) reflects the shift of the second receive signal as much as the estimated delay amount τ₁, the delay τ(t) of the second receive signal in the window w(t) can be determined by taking the estimated delay amount τ₁ into account. Thus, the delay in the window w(t−1) is determined as τ(t)=τ₁+τ₂.

An ultrasound elasticity image (i.e., strain image) is formed by considering the delay τ₂, which reflects the estimated delay amount τ₁ in the window w(t−1) corresponding to the relatively shallow region. Therefore, the error associated with the decorrelation of the first and second receive signals in the deep region can be decreased. In other words, the strain image is formed by reflecting the estimated delay amount τ₁ having a relatively low decorrelation and the delay τ₂ determined based on the estimated displacement τ_{1,} instead of using the delay τ(t) of the second receive signal as it is, which has relatively a high decorrelation.

A displacement x_{int pl}(t) of the tissue due to stress in window w(t) can be expressed as equation 11 below.

$${x}_{int pl} ( t ) = ( \frac{{τ}_{1}}{T} - ⌊ \frac{{τ}_{1}}{T} ⌋ ) {x}_{1} ( t + ⌈ {τ}_{1} / T ⌉ T ) + ( ⌈ \frac{{τ}_{1}}{T} ⌉ - \frac{{τ}_{1}}{T} ) {x}_{1} ( t + ⌊ {τ}_{1} / T ⌋ T ) \qquad ( Equation 11 )$$

In equation 11, “┌.┐” and “└.┘” denote the constants close to +28 and −∞ , respectively.

The delay τ(t) of the second receive signal at time ‘t’ in window w(t) can be obtained based on the estimated delay amount τ₁=τ(t−1) and the correlation of the displacement X_{int pl}(t) and the second receive signal x₂(t), as expressed in equation 12 below.

$$τ ( t ) = τ ( t - 1 ) + \frac{arg 〈 {x}_{int p} \cdot {x}_{2}^{*} 〉}{{ω}_{0} + {ω}_{B} ( t )} \qquad ( Equation 12 )$$

In equation 12, the second term is the delay τ₂ of the second receive signal in the window (t) with respect to the estimated delay amount τ₁. As shown in equation 12, the delay τ(t) in the window w(t) is determined by considering the instantaneous frequency ω_{B}(t) . Therefore, the errors due to the fixed center frequency ω₀ may be decreased. In other words, the error, which is associated with using the delay τ(t) as it is, can be reduced. This is because the delay τ(t) is determined in consideration of the delay τ₁=τ(t−1) in the window w(t−1) and the delay τ₂ in the window w(t) between the shifted signal and the non-shifted signal, which is expressed as the second term in equation 12. Therefore, the influence caused by the decorrelation can be reduced.

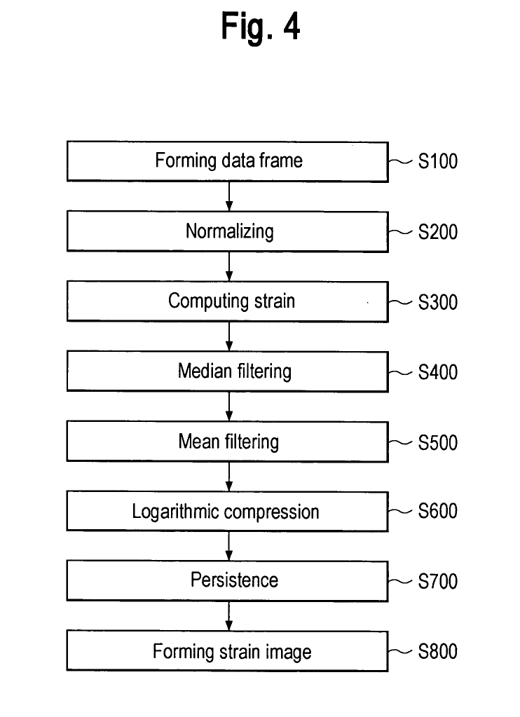

Now referring to FIG. 4, a method of forming ultrasound elasticity images will be described.

As shown in FIG. 4, the first and second receive signals in the RF form are demodulated and converted into I/Q baseband signals. Further, a data frame comprising I/Q baseband signals is formed (S100). I/Q baseband signals in the data frame are normalized (S200) and then the strain is computed with I/Q baseband signals (S300). Thereafter, a median filtering, a mean filtering, a logarithmic compression and persistence are performed one after the other (S400, S500, S600, S700). The strain images are then formed (S800).

The computation of strain by decreasing the decorrelation of the receive signals will be described below in detail.

The data frame is divided into a plurality of windows according to the depth of the tissues in the target subject or the lapse of time. A target window for decreasing the decorrelation and a reference window adjacent to the target window are selected among the plurality of windows. The target window and the reference window can be windows w(t) and w(t−1) of FIGS. 3a and 3b, respectively.

The delay amount is estimated in the reference window. One of the first and second receive signals in the target window is selected and shifted as much as the estimated delay amount. Afterwards, the instantaneous frequency and the delay are computed by using the correlation method. Thereafter, the strain is computed based on the delay.

As explained above, the instantaneous frequency changes depend on the depth of the tissues. Therefore, it can reduce the errors which occur when converting the phase value into the time value. Further, the shift of the receive signal increases the correlation, which results in the reduction of noises and the aliasing of phase.

While the present invention has been shown and described with respect to a preferred embodiment, those skilled in the art will recognize that various changes and modifications may be made without departing from the spirit and scope of the invention as defined in the appended claims.

What is claimed is:

1. A method of forming a strain image, comprising:
obtaining a first receive signal and a second receive signal, wherein the second receive signal is delayed with respect to the first receive signal;
computing a first correlation of the first receive signal and the second receive signal for a sampling period;
computing an instantaneous frequency based on the first correlation;
estimating a delay amount between the first and second receive signals;
selecting one of the first and second receive signals and shifting the selected signal as much as the estimated delay amount to reduce a delay between the first and second receive signals, wherein the non-selected signal remains as a non-shifted signal;
computing a second correlation of the non-shifted signal and the shifted signal;
obtaining a delay between the first and second receive signals based on the instantaneous frequency, the estimated delay amount and the second correlation;
obtaining a strain based on the delay; and
forming a strain image based on the strain.

2. The method of claim 1, wherein the first receive signal is obtained without applying stress to a target subject, and wherein the second receive signal is obtained with applying the stress to the target subject.

3. The method of claim 1, wherein the step of estimating the delay amount comprises the steps of:
dividing the first and second receive signals into a plurality of windows according to a depth of the target subject;
selecting a target window and a reference window in the plurality of windows, wherein the target window corresponds to a region deeper than the reference window; and
estimating the delay amount by comparing the first and second receive signals in the reference window.

4. The method of claim 3, wherein one of the first and second receive signals is selected and shifted in the target window, and wherein the delay in the target window is obtained.

5. A method of forming strain images, comprising:
transmitting an ultrasound signal to a target subject with or without applying stress to the target subject;
obtaining a first receive signal and a second receive signal, wherein the first receive signal is obtained without applying the stress to the target subject, and wherein the second receive signal is obtained with applying the stress to the target subject;
computing a first correlation of the first receive signal or the second receive signal for a sampling period;
computing an instantaneous frequency based on the first correlation;
estimating a delay amount between the first and second receive signals;
selecting one of the first and second receive signals and shifting the selected signal as much as the estimated delay amount to a direction of reducing a delay between the first and second receive signals, wherein the non-selected signal remains as a non-shifted signal;
obtaining a second correlation of the shifted signal and the non-shifted signal;
obtaining a delay between the first and second receive signals based on the instantaneous frequency, the estimated delay amount and the second correlation;
obtaining a strain based on the delay; and
forming the strain image based on the strain.

6. The method of claim 5, wherein the step of estimating the delay amount comprises the steps of:
dividing the first and second receive signals into a plurality of windows according to a depth of the target subject;
selecting a target window and a reference window in the plurality of windows, wherein the target window corresponds to a region deeper than the reference window; and
estimating the delay amount by comparing the first and second receive signals in the reference window.

7. The method of claim 6, wherein one of the first and second receive signals is selected and shifted in the target window, and wherein the delay in the target window is obtained.
